(19) United States
(12) Patent Application Publication
(10) Pub. No.: US 2012/0237553 A1
(43) Pub. Date: Sep. 20, 2012

(54) Process to Obtain a Homeopathic Medicament and Use Thereof

(75) Inventor: Lourival Brasil Felicio, Curitiba - PR (BR)

(73) Assignee: VALSANIC ENTERPRISES LLC, Wilmington, DE (US)

(21) Appl. No.: 13/395,812

(22) PCT Filed: Sep. 13, 2010
(86) PCT No.: PCT/BR2010/000307

§ 371 Date: Apr. 30, 2012

(30) Foreign Application Priority Data
Sep. 13, 2009 (IN) 2086/MUM/2009

(51) Int. Cl. A61K 36/74; A61P 1/16; A61P 35/02; A61P 35/00; A61P 37/00

(52) U.S. Cl. 424/278.1

(57) ABSTRACT

The present invention concerns a process to obtain a biological response modifier (BRM), particularly a medicament, as well as the medicament thus obtained and its use from highly diluted associations of vegetable, mineral and animal extracts, aiming to make use of its medicinal characteristics, internally and externally, in treatment, prevention, control and elimination of diseases, therapeutic and aesthetic finalities, in human beings.

The present invention concerns a process to obtain a biological response modifier (BRM), particularly a medicament, as well as the medicament thus obtained and its use from highly diluted associations of vegetable, mineral and animal extracts, aiming to make use of its medicinal characteristics, internally and externally, in treatment, prevention, control and elimination of diseases, and therapeutic and aesthetic finalities, in human beings.

### BACKGROUND OF THE INVENTION

The scientific advances in the last decade allowed an increase in the knowledge of physiopathology of several diseases, so that new modalities of immunotherapy are now used for several pathologies. The medicaments that modify the biological responses, stimulating the natural defense mechanisms of the host, are called “biological response modifiers”.

The efficacy of high dilutions of biologically active substances that alter immunologic response has been evaluated through different experimental and theoretical approaches. And the potentiated biological action of an association of vegetable and mineral extracts, even highly diluted, is justified by a synergistic action produced in multiple signaling biochemical routes.

Nowadays the attention of scientists and worldwide pharmaceutical industries aim to new forms of biological response modifying therapies with low toxicity for chronic diseases such as cancer, AIDS (Acquired Immunodeficiency Syndrome), hepatitis C, leukemias, etc. Those therapies are directed to specific cells or to cytokines that contribute to the immunoresponse. Thus, the production of a medicament obtained through the association of extracts of vegetal, mineral and animal origin, highly diluted, may denote a beneficial biological response, modulating the immunologic response, without citotoxicity, mutagenicity or mitogenicity.

Considering the immunologic system role in the tissue repair process (cicatrization), as well as in the homeostasis of oxygen reactive species, potentially involved in pathologic and cellular aging processes, the modulation of immunologic response promoted by the medicament of the invention is useful in the aesthetics area, both for promoting tissue repair and anti-aging action.

### DESCRIPTION OF THE INVENTION

The present invention concerns in a first aspect the process to obtain a medicament through several steps of dilution and dynamization of several solutions, associated in different stages.

To facilitate the present process, prior to the several stages of association of the multiple components, one may prepare the dynamized decimal dilutions of the several vegetable and mineral mother tinctures, as well as the Lachesis animal dilution in its tenth dynamized decimal dilution.

Thus, besides the dilution of Lachesis, the following vegetable-origin solutions are previously prepared:

a) the fifth dynamized decimal solution from mother tinctures of the vegetables Conium maculatum, Bryonia alba, Pulsatilla, Ipecacuana and Riccinus;
b) the sixth dynamized decimal solution from the mother tincture of the vegetables Rhus toxicus and Thuya occidentalis; and
c) the mother tinctures of the vegetables Asa foetida and Aconitum napellus.

The following solutions of mineral origin are also previously prepared:

a) the sixth dynamized decimal dilution from Arsenicum album;
b) the twelfth dynamized decimal solution of Phosphorus, Silicia and Sulphur;
c) the fifth dynamized centesimal dilution of calcium carbonate.

The process of the invention is characterized by the following steps:

a) associate 25 ml of the fifth dynamized centesimal dilution of calcium carbonate with 75 ml of 70% alcohol; succuss it;
b) add 10 ml Asa foetida mother tincture, 10 ml Aconitum mother tincture, 15 ml of the prior step (a) composition and 65 ml of 70% alcohol; succuss it;
c) to 10 ml of prior step (b) composition, add 25 ml of the fifth dynamized centesimal dilution of calcium carbonate and 65 ml of 70% alcohol; succuss it;
d) to 10 ml of the prior step (c) composition, add 25 ml of the fifth dynamized centesimal dilution of calcium carbonate and 65 ml of 70% alcohol; succuss it;
e) to 10 ml of the prior step (d) composition, add 25 ml of the fifth dynamized centesimal dilution of calcium carbonate and 65 ml of 70% alcohol; succuss it;
f) to 10 ml of the prior step (e) composition add 10 ml of the fifth dynamized centesimal dilution of calcium carbonate and 80 ml 70% alcohol; succuss it;
g) to 10 ml of the prior step (f) composition add 90 ml of 70% alcohol, obtaining a first composition; succuss it;
h) dilute 10 ml of the prior step (g) composition in 90 ml of 70% alcohol; succuss it;
i) in 10 ml of the prior step (h) composition add 10 ml of the twelfth dynamized decimal dilution of Sulphur and 80 ml of 70% alcohol, obtaining a second composition; succuss it;
j) add 10 ml of the sixth dynamized decimal dilution of Arsenicum album, 10 ml of the sixth dynamized decimal dilution of Rhus toxicus, 10 ml of the prior step (i) composition and 70 ml of 70% alcohol; succuss it;
k) associate 10 ml of the sixth dynamized decimal dilution of Thuya occidentalis, 10 ml of the fifth dynamized decimal dilution of Conium maculatum, 10 ml of the prior step (j) composition and 70 ml of 70% alcohol; succuss it;
l) associate 10 ml of the fifth dynamized decimal dilution of Bryonia alba, 10 ml of the fifth dynamized decimal dilution of Riccinus, 10 ml of the prior step (k) composition and 70 ml of 70% alcohol; succuss it;
m) associate 10 ml of the fifth dynamized decimal dilution of Pulsatilla, 10 ml of the prior step (l) composition, 10 ml of the fifth dynamized decimal dilution of Ipecacuana and 70 ml distilled water; succuss it;
n) associate 1 ml of the tenth dynamized decimal dilution of Lachesis, 10 ml of the twelfth dynamized decimal dilution of Phosphorus, 10 ml of the prior step (m) composition and 79 ml of 70% alcohol; succuss it;
o) associate 1 ml of the twelfth dynamized decimal dilution of Silicia, 10 ml of the prior step (n) composition and 89 ml of 70% alcohol; succuss it;
p) associate 5 ml of the prior step (o) composition, 5 ml of step (f) composition and 40 ml of 70% alcohol; succuss it;
q) dilute 5 ml of the prior step (p) composition in 10 ml of 96% alcohol and 35 ml distilled water; succuss it;
r) dilute 5 ml of the prior step (q) composition in 10 ml of 96% alcohol and 35 ml distilled water; succuss it; and
s) dilute the composition of step (r) another 3 times in the 1:10 proportion with distilled water, and succuss it every time.

Succussion, according the meaning employed herein, means vigorous agitation of the flask where dilution or mixture is performed, with 100 strokes against a semi-rigid shield. The succcussion may be manual or mechanized.

The alcohol used in the invention is neutral. All tinctures and dilutions employed are prepared according to the methodologies described in the German Homeopathic Pharmacopeia.

Another aspect of the invention relates to a medicament prepared according to the steps (a) to (s) above described, which may be considered a homeopathic medicament, due to the following characteristics:

a. the medicament is dynamized, that is, it is obtained by diluting its components in decimal scale and is succussioned, characterizing the hannemanian methodology;

b. it is prepared from animal, vegetal and mineral substances;

c. it does not present toxicity and side reactions, as all pharmacological actives are highly diluted;

d. the homeopathic medicament is complex, as it is comprises more than one single medicament, administrated simultaneously (complexist homeopathy practice);

Nevertheless, this medicament presents innovative characteristics:

a) Novel method of production in several specific steps of associations, and the originality of such associations in homeopathic matrices.

b) The first association is obtained in such a way that each dynamization (decimal dilution followed by succussion) the mixture receives a new addition of the fifth centesimal dilution of calcium carbonate. At this stage there are two great differentials:
a decimal dilution of a matrix in centesimal scale, not previewed by any pharmacopeia.
considering that with each dynamization (dilution and succussion) energetic conditions of a medicament are modified, the progression of this dynamization scale always receiving a fifth centesimal dilution matrix will produce a growing dynamization scale of that matrix, so it can be inferred that such a substance (calcium carbonate) presents itself in this medicament as an “energetic chord”, that is, in various simultaneous stages of dynamization.

The present invention provides a new modifier of the biological response. Biologic assays, in vitro and in vivo, showed that this new formulation acts as a BRM—biological response modifier—as it can activate macrophages and after treatment reactive products of oxygen and nitrogen (ROS and RNS) can be detected and quantified. This formulation also induces increase of B cell precursors in bone marrow and increase of TCD8+ in lymph node when added in drinking water for seven days.

### Example

Macrophages were washed from peritoneal cavities with 10 ml of cold Phosphate Buffer Solution (PBS), at pH 7.4. The macrophages were incubated at 37° C. under 5% CO₂ for 15 min. and the non-adherent cells were removed by washing with PBS. Dulbecco's Modified Eagle's Medium (DMEM) supplemented with 10% Fetal Bovine Serum (FBS), 50 μg/ml penicillin and 100 U/ml gentamicin were added to the culture. Cultured cells were treated with this formulation (200 μl/ml), and after 24 h a new dose of 20 μl/ml was given without replacing the medium. Treatment was carried out for 48 h in vitro. The formulation was always vigorously shaken, with the process named succussion, immediately before treatment.

Significant differences were observed in the treated group when compared to control groups (cells not treated with the formulation of the invention). Cells from the control groups were mainly resident macrophages, about 70%, and few activated macrophages were also present. Almost all cells from the treated group were activated, as defined by morphological alterations. About 86% of treated macrophages were activated when observed in light microscopy and transmission electron microscopy. The increased response capacity of activated macrophages is a result, in part, of the increase capacity of these cells to produce oxygen radicals; thus the oxidative metabolism of treated macrophages was evaluated, as described in the sequence.

Reduction of ferricytochrome c was used to measure rates of formation of O₂⁻ in culture supernatant. For measurement of O₂⁻, cells (5×10⁵ cells/well) were incubated in 80 μM HBSS containing ferricytochrome c (commercialized by Sigma Chemical Co.) in the presence or absence of 1 μg/ml phorbol miristate acetate (PMA). Since PMA is able to induce O₂⁻ production by macrophages it was used as positive control. Absorbance was measured at 550 nm in a microplate reader (commercialized by BIO-RAD Laboratories) and the extinction molar coefficient ε=2.1×10⁴ M⁻¹ Ca⁻¹ was used to determine reduced ferricytochrome c. Results are expressed as nmol O₂⁻/10⁶ cells.

After 15 min a significant decreased liberation of O₂⁻ in the culture supernatant was found:

| | Mean | Standard Deviation |
|---|---|---|
| Control | 17.1 | 0.6 |
| Control + PMA | 17.7* | 0.6 |
| Med 8 | 16.2* | 0.6 |
| Med 8 + PMA | 16.9 | 0.4 |

Med 8 is the code for this formulation and * shows when the difference is significant when compared with the respective control (*= P < 0.05).

Production of Hydrogen Peroxide (H₂O₂) by macrophages after treatment was quantified based on the horseradish peroxidase-dependent oxidation of phenol red by H₂O₂⁵. Macrophages (3.5×10⁵ cells/well) were incubated in 15 U/ml, type IV-A horseradish peroxidase (commercialized by Sigma Chemical Co.) and 194 mg/ml phenol red solution dissolved in HBSS at 4° C., briefly before the start of the experiment. 1 μg/ml of PMA was added as a positive control of H₂O₂ production²⁶. The plates were incubated at 37° C. for the desired time interval (60 and 90 min) and the reaction stopped by adding 10 μl 1M NaOH aqueous solution per well. The absorbance of cell-free culture supernatant was read at 620 nm at a microplate reader (SLT Lab Instruments 340 ATC). The H₂O₂ concentration was determined by reference to a standard curve using 1-50 nmol of H₂O₂ in a solution containing 15 U/ml peroxidase, 194 mg/ml phenol red in HBSS. A significant decreased liberation of H₂O₂ in the culture supernatant was found:

| | Mean | Standard Deviation |
|---|---|---|
| Control | 16.97 | 0.93 |
| Control + PMA | 17.28 | 2.75 |
| Med 8 | 15.39* | 0.81 |
| Med 8 + PMA | 16.31 | 0.39 |

Med 8 is the code for this formulation and * shows when the difference is significant when compared with the respective control (*= P < 0.05).

The NO generation was estimated by sampling culture supernatants for nitrite, which is a stable product of NO reaction. Macrophages treated in vivo and in vitro (5×10⁵ cells/well) were plated into 96-well tissue culture plates. After 48 h, aliquots of 100 μl of cell-free supernatant were mixed with an equal volume of Griess-reagent (0.5% sulfanilamide and 0.05% N-1-naphtyl ethylenediamine dihydrochloride in 2.5% phosphoric acid) in 96-well tissue culture plates and incubated for 10 min at 25° C. 50 ng/ml LPS and 26 U/ml IFN-γ were added as a positive control for NO production. Optic density of the samples was subsequently measured at 550 nm at a microplate reader (commercialized by BIO-RAD Laboratories). The nitrite concentration was determined by reference to a standard curve using sodium nitrite (10-80 μM) diluted in culture medium. A significant increased liberation of NO in the culture supernatant was found:

| | Mean | Standard Deviation |
|---|---|---|
| Control | 28.4 | 1.4 |
| Control + LPS/IFNy | 35.0* | 1.9 |
| Med 8 | 31.9* | 2.6 |
| Med 8 + LPS/IFNγ | 32.3* | 2.0 |

Med 8 is the code for this formulation and * shows when the difference is significant when compared with the respective control (*= P < 0.05).

After 96 hours, the supernatant of plated culture was centrifuged and the quantification of cytokines measured by mouse Th1/Th2 cytokine CBA (Cytometric Bead Array) kit (commercialized by BD Pharmingen), according to the manufacturer instructions. This kit contains antibodies against IL-2, IL-4, IL-5, INF-γ and TNF-α cytokines. Cytokine concentration was obtained comparing data with a cytokine curve in the CBA program (commercialized by Becton Dickinson). Fluorescence was measured by a FACSCalibur flow cytometer (commercialized by Becton Dickinson), equipped with an argon ion laser (488 nm). Data was analyzed in a program commercialized by Cell Quest, according to manufacturer procedures. Data analysis was performed with ANOVA and Tukey test (P<0.05) to determine the statistical significance of the intergroup comparisons. So, if the cells are producing excess of TNFα, this formulation can reduce it.

| Group | Number | total | Mean |
|---|---|---|---|
| Control | 4 | 408.35 | 102.09 |
| Med 8 | 4 | 364.82 | 91.20* |

Med 8 is the code for this formulation and * shows when the difference is significant when compared with the respective control (*= P < 0.05).

Nitric Oxide (NO) reacts very rapidly with oxygen radicals. The chemical and biological interaction of NO and ROS with various biological molecules has important consequences in the mechanisms of different immunological and pathological conditions. Macrophages have the opportunity to produce O₂⁻ and NO in nearly equimolar amounts. As NO migrates near to the source of O₂⁻, it reacts to form peroxynitrite (ONOO⁻). Thus the primary chemistry of ONOO⁻ would be within close proximity of the O₂⁻ source. Without being bound by theory, as NO is small and uncharged, it can traverse the vesicle membrane and it can be assumed that in macrophages treated with this formulation ONOO⁻ formation would be occurring within vesicles. It can be supported by the fact that O₂⁻ release from treated cells diminished considerably after 15 min.

Femurs from three-month old swiss mice were dissected and cleaned. Epiphyses were removed and the marrow was flushed with Dulbecco's Modified Eagle Medium (DMEM) containing 10% fetal bovine serum with 1 U/ml penicillin, 1 μg/ml streptomycin and 2.5 μg/ml amphoterycin. Cells were counted in a Neubauer chamber and adjusted depending of the experiments. They were seeded in 24- or 96-well culture plates or into culture flasks, and maintained at 37° C. in 5% CO₂ atmosphere for 24, 48, 72 and 96 hours.

Lymph nodes were removed from the mesentery. The tissue was dissociated using sterile Medicons (commercialized by Becton Dickinson) and the cell suspension was filtered with Filcons (commercialized by Becton Dickinson)/100 μm mesh to remove tissue fragments. After washing by centrifugations, the final suspension was incubated in a culture flask with PBS at 37° C. in a humidified atmosphere containing 5% CO₂. After 40 min of incubation, the non-adherent cell suspension was transferred to sterile tubes, washed three times with PBS and the cell number was determined using an automated cell counter (commercialized by CELM-Brazil). These cells were plated and cultured for each experiment.

Surface markers were determined using a flow cytometer. Cells (10⁶) were fixed with 1% paraformaldehyde, washed, counted and incubated with a biotinylated antibody (0.5 μg/ml) against CD45R (lymphocyte B marker), CD11b (Mac-1) (monocytes/macrophage marker), CD11c (dendritic cells marker), CD3 (lymphocyte T marker), Ly-6G (granulocyte marker) and TER-119 (erythrocyte marker) in PBS for 40 minutes. After that they were washed with PBS and incubated with 0.5 μg/ml phycoerythrin (PE) labeled secondary antibody in PBS for 30 minutes. Fluorescence was analyzed according to standard procedures on a FACSCalibur flow cytometer (commercialized by Becton Dickinson), equipped with an argon ion laser (488 nm). Data were analyzed in Cell Quest program (commercialized by Becton Dickinson).

Data was submitted to analysis of variance with factorial diagram (2×3) to determine the statistical significance. The Tukey test was performed when the effects of interaction were significant. The level of significance was taken at p<0.05. Data is representative of three independent experiments. Accordingly, this formulation induces an increase of B cell precursors in bone marrow and an increase of TCD8+ in lymph node.

So the use of the invention gently increases both the innate and acquired immune response, activating macrophages in a non-classic way.

A person skilled in the art may be able to reduce the invention to practice with the aid of the teachings contained herein, in ways not exactly as described, but it is understood that different embodiments that perform substantially the same function to reach substantially the same result of the invention are equivalent realizations also covered by the attached claims.

1. A process for obtaining a biological material response modifier, comprising the steps of:
a) associate 25 ml of the fifth dynamized centesimal dilution of calcium carbonate to 75 ml of 70% alcohol; succuss it;
b) add 10 ml Asa foetida mother tincture, 10 ml Aconitum mother tincture, 15 ml of the prior step (a) composition and 65 ml of 70% alcohol; succuss it;
c) to 10 ml of prior step (b) composition, add 25 ml of the fifth dynamized centesimal dilution of calcium carbonate and 65 ml of 70% alcohol; succuss it;
d) to 10 ml of the prior step (c) composition, add 25 ml of the fifth dynamized centesimal dilution of calcium carbonate and 65 ml of 70% alcohol; succuss it;
e) to 10 ml of the prior step (d) composition, add 25 ml of the fifth dynamized centesimal dilution of calcium carbonate and 65 ml of 70% alcohol; succuss it;
f) to 10 ml of the prior step (e) composition add 10 ml of the fifth dynamized centesimal dilution of calcium carbonate and 80 ml 70% alcohol; succuss it;
g) to 10 ml of the prior step (f) composition add 90 ml of 70% alcohol, obtaining a first composition; succuss it;
h) dilute 10 ml of the prior step (g) composition in 90 ml of 70% alcohol; succuss it;
i) in 10 ml of the prior step (h) composition add 10 ml of the twelfth dynamized decimal dilution of Sulphur and 80 ml of 70% alcohol, obtaining a second composition; succuss it;
j) add 10 ml of the sixth dynamized decimal dilution of Arsenicum album, 10 ml of the sixth dynamized decimal dilution of Rhus toxicus, 10 ml of the prior step (i) composition and 70 ml of 70% alcohol; succuss it;
k) associate 10 ml of the sixth dynamized decimal dilution of Thuya occidentalis, 10 ml of the fifth dynamized decimal dilution of Conium maculatum, 10 ml of the prior step (j) composition and 70 ml of 70% alcohol; succuss it;
l) associate 10 ml of the fifth dynamized decimal dilution of Bryonia alba, 10 ml of the fifth dynamized decimal dilution of Riccinus, 10 ml of the prior step (k) composition and 70 ml of 70% alcohol; succuss it;
m) associate 10 ml of the fifth dynamized decimal dilution of Pulsatilla, 10 ml of the prior step (l) composition, 10 ml of the fifth dynamized decimal dilution of Ipecacuana and 70 ml distilled water; succuss it;
n) associate 1 ml of the tenth dynamized decimal dilution of Lachesis, 10 ml of the twelfth dynamized decimal dilution of Phosphorus, 10 ml of the prior step (m) composition and 79 ml of 70% alcohol; succuss it;
o) associate 1 ml of the twelfth dynamized decimal dilution of Silicia, 10 ml of the prior step (n) composition and 89 ml of 70% alcohol; succuss it;
p) associate 5 ml of the prior step (o) composition, 5 ml of step (f) composition and 40 ml of 70% alcohol; succuss it;
q) dilute 5 ml of the prior step (p) composition in 10 ml of 96% alcohol and 35 ml distilled water; succuss it;
r) dilute 5 ml of the prior step (q) composition in 10 ml of 96% alcohol and 35 ml distilled water; succuss it; and
s) dilute the composition of step (r) another 3 times in the 1:10 proportion with distilled water, and succuss it every time.

2. The process according to claim 1, wherein the process is preceded by preparation of a solution selected from the group consisting of: Conium maculatum in its fifth dynamized decimal dilution from the mother tincture; Bryonia alba in its fifth dynamized decimal dilution from the mother tincture; Pulsatilla in its fifth dynamized decimal dilution from the mother tincture; Ipecacuana in its fifth dynamized decimal dilution from the mother tincture; Riccinus in its fifth dynamized decimal dilution from the mother tincture; Rhus toxicus in its sixth dynamized decimal dilution from the mother tincture; Thuya occidentalis in its sixth dynamized decimal dilution from the mother tincture; Conium maculatum in its fifth dynamized decimal dilution from the mother tincture; Phosphorus in its twelfth dynamized decimal dilution; Silicia in its twelfth dynamized decimal dilution; Sulphur in its twelfth dynamized decimal dilution; Phosphorus in its twelfth dynamized decimal dilution; Calcium carbonate in its fifth dynamized centesimal dilution; and combinations thereof.

3. A biological response modifier obtained by the association of highly diluted vegetal, animal and mineral extracts.

4. A biological response modifier according to claim 3 obtained by the process according to claim 1.

5-9. (canceled)

10. A biological response modifier according to claim 3 obtained by the process according to claim 2.

11. A method to induce decrease in the production of TNFα produced in excess by administering to a patient in need a biologic response modifier medicament according to claim 3.

12. A method to induce decrease in the production of TNFα produced in excess by administering to a patient in need a biologic response modifier medicament according to claim 4.

13. A method to induce decrease in the production of TNFα produced in excess by administering to a patient in need a biologic response modifier medicament according to claim 10.

14. A method to induce increase in B cell precursors in bone marrow by administering to a patient in need a biologic response modifier medicament according to claim 3.

15. A method to induce increase in B cell precursors in bone marrow by administering to a patient in need a biologic response modifier medicament according to claim 4.

16. A method to induce increase in B cell precursors in bone marrow by administering to a patient in need a biologic response modifier medicament according to claim 10.

17. A method to induce increase of TCD8+ in lymph node by administering to a patient in need a biologic response modifier medicament according to claim 3.

18. A method to induce increase of TCD8+ in lymph node by administering to a patient in need a biologic response modifier medicament according to claim 4.

19. A method to induce increase of TCD8+ in lymph node by administering to a patient in need a biologic response modifier medicament according to claim 10.

20. A method for treating a disease selected from the group consisting of: cancer, AIDS, leukemia and hepatitis C; by administering to a patient in need a biologic response modifier medicament according to claim 3.

21. A method for treating a disease selected from the group consisting of: cancer, AIDS, leukemia and hepatitis C; by administering to a patient in need a biologic response modifier medicament according to claim 4.

22. A method for treating a disease selected from the group consisting of: cancer, AIDS, leukemia and hepatitis C; by administering to a patient in need a biologic response modifier medicament according to claim 10.
